(19) United States
(12) Patent Application Publication
(10) Pub. No.: US 2010/0036239 A1
(43) Pub. Date: Feb. 11, 2010

(54) PROCEDURE TO PLAN, GUIDE AND ASSESS PERCENTANEOUS TRANSLUMINAL HEART VALVE REPAIR

(76) Inventor: Klaus Klingenbeck-Regn, Nurnberg (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/187,514

(22) Filed: Aug. 7, 2008

(51) Int. Cl. A61B 6/03; A61F 2/24

(52) U.S. Cl. 600/425; 623/2.11

(57) ABSTRACT

A method or workflow for heart valve emplacement provides a planning stage in which the patient is imaged using a C-arm computed tomography-like image system to obtain a electrocardiogram gating image that is measured to determine a size of a prosthetic heart valve for use on the patient. A deployment stage provides that the selected heart valve prosthesis is inserted into the patient via minimally invasive procedures and is positioned at the location where it is to be anchored, and while still in a non-expanded state an image is obtained to determine if the prosthesis is properly positioned for expansion. The prosthesis is expanded so that it becomes anchored in position. In an assessment stage, an image is obtained to determine if the expanded valve prosthesis is in the desired position. If necessary, the prosthesis is moved or further expanded. A final assessment image may be obtained to check the final position.

### BACKGROUND OF THE INVENTION

1. Field of the Invention

The present invention relates generally to a method for heart valve repair or replacement, and more specifically to a workflow for imaging during a heart valve replacement procedure.

2. Description of the Related Art

Heart problems may be caused by a number of different possible sources. Among the possible sources of heart problems is problems with one or more of the valves of the heart. The heart valves may become damaged or diseased. A treatment for heart valve damage or disease is replacement of the heart valve. Heart valve treatment has previously involved open chest surgery to expose the heart to the surgeon.

Minimally invasive surgery techniques are being used more commonly in an ever widening number of surgical procedures. Minimally invasive repair or replacement of heart valves is becoming possible. Medical imaging is of critical importance in successful repair or replacement of a heart valve or other interventional procedures. The medical imaging systems can produce a two dimensional image or a three dimensional image of the patient, or to be more precise of a portion of the patient. Computed tomography (CT) is one of the many techniques of imaging within the body for such procedures.

CT-like 3D-imaging using C-arm systems inside the angiography lab has been introduced into interventional radiology. An example of a C-arm system is made by Siemens AG, and uses a technique named Syngo Dyna CT to provide CT-like images in an angiographic computed tomography technique. The application of this type of system will be extended to interventional cardiology and electrophysiology by complementing the Dyna CT data acquisition and reconstruction capability with ECG (ElectroCardioGram) gating to avoid motion artifacts due to the beating heart, in other words, imaging the heart using techniques to freeze heart motion to a specific phase of the heart cycle. With a Dyna CT ECG system it will be possible to image cardiac structures and anatomy close to the heart in a 3D-fashion. Percentaneous Transluminal Valve Repair (PTVR) is an upcoming minimally invasive procedure to replace heart valves while avoiding the burden of surgery. Due to the complex and patient specific anatomy of the heart in the area of the heart valves, good 3D-imaging is essential to optimize the clinical outcomes.

### SUMMARY OF THE INVENTION

The present invention provides a method for performing a repair of a heart valve using minimally invasive procedures, including a planning stage in which imaging of the heart valves is performed to determine a size of replacement valve to be used, a deployment stage in which the replacement valve is moved into position by minimally invasive techniques and the position is checked using the imaging system prior to expansion of the prosthesis in place, and an assessment stage in which an image is obtained of the expanded prosthesis to ensure proper positioning.

### BRIEF DESCRIPTION OF THE DRAWINGS

FIG. 1 is a process flow chart showing the steps of a preferred embodiment of the method for heart valve placement according to the principles of the present invention.

### DETAILED DESCRIPTION OF THE PREFERRED EMBODIMENTS

The present invention is disclosed with respect to a C-arm medical imaging system, and in particular to the Siemens Dyna CT ECG system. The method disclosed herein is applicable to other medical imaging systems as well.

Medical personnel have determined that the patient has heart disease and requires replacement of a heart valve with an artificial heart valve. Permission is obtained to perform the heart procedure and the patient is prepared.

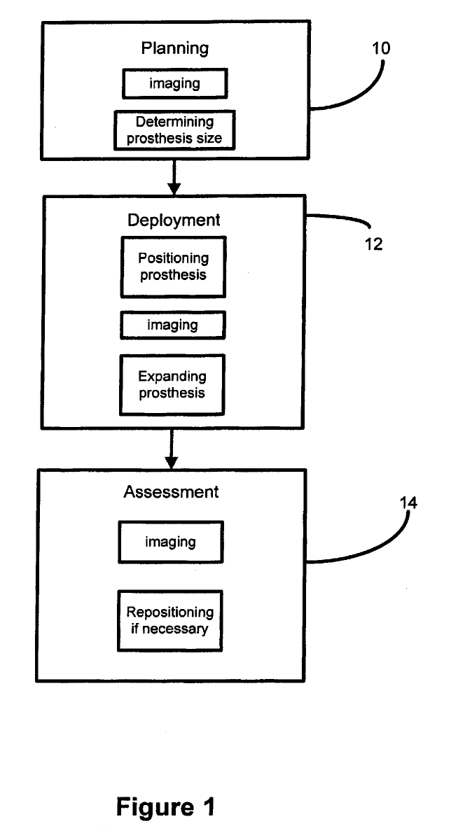

1) Planning—as shown at 10 in FIG. 1

In a first stage of the present method or workflow, planning of the valve repair is performed. A determination must be made as to which replacement valve will best fit the particular patient.

Different kinds of valve prostheses are available with different sizes and in particular in different diameters. Pre-procedural 3D-imaging is performed with a Dyna CT ECG imaging system or other imaging system to determine the proper size of replacement valve for each patient individually. For this step, a dye is administered to the patient in such a way as to enhance the aorta, the aortic root and the cardiac chambers of the patient. Imaging of the cardiac region of the patient while the dye is in place is carried out to obtain a high contrast image of the heart structures. The imaging data is processed using computed tomography methods to obtain a three-dimensional image of the patient. Software tools are made available that in conjunction with the imaging data is capable of measuring the aorta at different levels. Automatic segmentation tools are provided to segment the images of the structural elements of interest (aorta, atria, ventricles, etc.). This way the desired diameter and length of the valve prosthesis is determined.

2) Deployment—as shown at 12 in FIG. 1

In the next stage of the method or workflow, the valve prosthesis that has been selected is emplaced in the patient. The prosthesis valve is an expandable structure that is inserted into the body and moved into position while in a non-expanded, or compressed, state. The prosthesis is advanced to the desired location using a catheter inserted through the groin and along the femoral vessels. Once the prosthesis is at the desired position, the Dyna CT ECG imaging system can be used to check the proper position for the prosthesis before deployment, in other words, before expansion of the balloon or self-expansion of a self expanding prosthesis. Alternatively, the position of the prosthesis can be derived from fluoroscopy through a 2D/3D image data registration with the 3D image data set from the planning step 1.

After the prosthesis position has been determined to be correct, the prosthesis is expanded into place. Expansion of the prosthesis anchors the prosthesis into position in the patient's body.

The important question is how well the prosthesis fits, for example is the prosthesis in tight apposition to the atrial wall.

3) Assessment—as shown at 14 in FIG. 1

After expansion of the prosthesis, additional 3D imaging is performed to check the apposition of the prosthesis. The assessment imaging is preferably also performed by the Dyna CT imaging system. If apposition is not perfect, further balloon expansion can be applied until a perfect fit is achieved.

In order to facilitate the assessment of the prosthesis position, image slices taken orthogonal to the center line of the prosthesis are generated from the imaging data obtained by the imaging system and are displayed automatically. After a first correction—if needed—another 3D imaging step by the Dyna CT system for positional assessment can be performed.

The previous description has used the aortic valve as an example. The same applies to the other cardiac valves, like the mitral valve and tricuspid valve.

Virtual models of the various kinds of prostheses can be stored in the computer and can be used to simulate the delivery of the prosthesis into position at the end of step 1 once the patient's 3D data set is available. The best fit model of prosthesis can be selected either by visual inspection or by automatic and quantitative evaluation. A quantitative measure could be the mean or mean square distance between all struts of the prosthesis and the aortic wall. Open “holes” around the prosthesis cause regurgitation and detonate the pumping function through the valve and are to be avoided.

When ECG gating is applied at different points in time during the heart cycle functional information about heart motion can be obtained. This can be used pre- and post-interventional to check how far function has been restored. By automatic segmentation of the chambers endsystolic and enddiastolic volumes can be calculated, to evaluate the ejection fractions which are an important parameter to assess cardiac function.

Thus, there is shown and described a method or workflow for emplacement of a heart valve prosthesis which provides a planning stage in which the patient is imaged using a C-arm computed tomography-like image system to obtain a electrocardiogram gating image that is measured to determine a size of a prosthetic heart valve for use on the patient. A deployment stage provides that the selected heart valve prosthesis is inserted into the patient via minimally invasive procedures and is positioned at the location where it is to be anchored, and while still in a non-expanded state an image is obtained to determine if the prosthesis is properly positioned for expansion. The prosthesis is expanded so that it becomes anchored in position. In an assessment stage, an image is obtained to determine if the expanded valve prosthesis is in the desired position. If necessary, the prosthesis is moved or further expanded. A final assessment image may be obtained to check the final position.

The imaging system is used at each stage of the prosthesis emplacement to determine the proper size of prosthesis, to determine the position of the prosthesis prior to expansion, and to check the position of the prosthesis after expansion. The doctor performing the installation of the prosthesis has better information available before, during and after the procedure, and the patient has a better result.

Although other modifications and changes may be suggested by those skilled in the art, it is the intention of the inventors to embody within the patent warranted hereon all changes and modifications as reasonably and properly come within the scope of their contribution to the art.

We claim:

1. A method for emplacement of a heart valve prosthesis, comprising the steps of:
planning emplacement of a heart valve prosthesis in a patient, including the sub-steps of:
imaging a cardiac region of the patient using a computed tomography imaging system,
determining a size of heart valve prosthesis for emplacement;
deploying a heart valve prosthesis of the determined size, including the sub-steps of:
inserting the heart valve prosthesis into the body of the patient in a non-expanded state,
moving the heart valve prosthesis into a position in the heart of the patient for emplacement,
imaging the cardiac region of the patient to check for proper positioning of the non-expanded prosthesis,
expanding the heart valve prosthesis to anchor the prosthesis in position; and
assessing a position of the heart valve prosthesis in an expanded position, including the sub-steps of:
imaging the cardiac region of the patient to determine an anchored position of the prosthesis, and
adjusting a position of the prosthesis if necessary.

2. A method as claimed in claim 1, further comprising the step of:
in said assessing step, further imaging the cardiac region of the patient after said adjusting step.

3. A method as claimed in claim 1, wherein said imaging steps are performed by a C-arm imaging system.

4. A method as claimed in claim 3, wherein said C-arm imaging system uses electrocardiogram gating image techniques.

5. A method as claimed in claim 1, wherein said step of planning includes a sub-step of using a dye to increase image contrast during the imaging sub-step.
